(19) United States
(12) Patent Application Publication
(10) Pub. No.: US 2026/0090743 A1
(43) Pub. Date: Apr. 2, 2026

(54) PULSE OXIMETER

(71) Applicant: SEIKO EPSON CORPORATION, Tokyo (JP)

(72) Inventor: Akira IKEDA, CHINO-SHI (JP)

(73) Assignee: SEIKO EPSON CORPORATION, Tokyo (JP)

(21) Appl. No.: 19/337,944

(22) Filed: Sep. 24, 2025

(30) Foreign Application Priority Data
Sep. 27, 2024 (JP) 2024-168935

(51) Int. Cl. A61B 5/1455; A61B 5/00; A61B 5/11

(52) U.S. Cl. CPC A61B 5/14552; A61B 5/1126; A61B 5/7235; A61B 5/7271; A61B 5/742; A61B 2562/0238

(57) ABSTRACT

A pulse oximeter includes: a red light sensor detecting a red light from a subject; an infrared light sensor detecting an infrared light from the subject, and a control device configured to process detection signals from the red light sensor and the infrared light sensor. The control device includes an oxygen saturation measurement unit for generating an oxygen saturation data group based on the detection signals, and an abnormal value detection unit for detecting an abnormal value of the oxygen saturation data group based on a body motion of the subject based on at least one of a red light signal intensity ratio of an amplitude signal of the detection signal from the red light sensor to a signal intensity and an infrared light signal intensity ratio of an amplitude signal of the detection signal from the infrared light sensor to a signal intensity.

The present application is based on, and claims priority from JP Application Serial Number 2024-168935, filed Sep. 27, 2024, the disclosure of which is hereby incorporated by reference herein in its entirety.

### BACKGROUND

### 1. Technical Field

The present disclosure relates to a pulse oximeter.

### 2. Related Art

A pulse oximeter measures an arterial blood oxygen saturation (SpO2) by irradiating a finger with red light and near infrared light sources and detecting light transmitted through the finger (see JP-A-2004-337605). When an oxygen desaturation index, which is an indicator of sleep apnea syndrome, is tested, the arterial blood oxygen saturation of a subject during sleep is measured by the pulse oximeter.

JP-A-2004-337605 is an example of the related art.

When the arterial blood oxygen saturation of the subject during sleep is measured, a disturbance signal (noise component) is mixed into a detection signal based on detection light in the pulse oximeter due to a body motion of the subject, and thus there is a problem that measurement accuracy of the oxygen saturation measured based on the detection signal deteriorates.

### SUMMARY

A pulse oximeter according to a first aspect of the disclosure includes: a red light sensor configured to irradiate a subject with red light and detect the red light transmitted through or reflected from the subject; an infrared light sensor configured to irradiate the subject with infrared light and detect the infrared light transmitted through or reflected from the subject; and a control device configured to process detection signals from the red light sensor and the infrared light sensor, in which the control device includes an oxygen saturation measurement unit for generating, based on the detection signals from the red light sensor and the infrared light sensor, an oxygen saturation data group indicating an oxygen saturation of the subject, and an abnormal value detection unit for detecting an abnormal value of the oxygen saturation data based on a body motion of the subject based on at least one of a red light signal intensity ratio indicating a ratio of an amplitude of a vibration component in the detection signal output by the red light sensor to a signal intensity of the detection signal and an infrared light signal intensity ratio indicating a ratio of an amplitude of a vibration component in the detection signal output by the infrared light sensor to a signal intensity of the detection signal.

A pulse oximeter according to a second aspect of the disclosure includes: a red light sensor configured to irradiate a subject with red light and detect the red light transmitted through or reflected from the subject; an infrared light sensor configured to irradiate the subject with infrared light and detect the infrared light transmitted through or reflected from the subject; a control device configured to process detection signals from the red light sensor and the infrared light sensor; and a display unit configured to display information obtained by the control device, in which the control device generates, based on the detection signals from the red light sensor and the infrared light sensor, an oxygen saturation data group indicating an oxygen saturation of the subject, calculates an indicator value based on at least one of a red light signal intensity ratio indicating a ratio of an amplitude of a vibration component in the detection signal output by the red light sensor to a signal intensity of the detection signal and an infrared light signal intensity ratio indicating a ratio of an amplitude of a vibration component in the detection signal output by the infrared light sensor to a signal intensity of the detection signal, and displays, on the display unit, a graph in which the oxygen saturation data group and the indicator value are displayed on the same time axis.

A pulse oximeter according to a third aspect of the disclosure includes: a red light sensor configured to irradiate a subject with red light and detect the red light transmitted through or reflected from the subject; an infrared light sensor configured to irradiate the subject with infrared light and detect the infrared light transmitted through or reflected from the subject; a control device configured to process detection signals from the red light sensor and the infrared light sensor; and a display unit configured to display information obtained by the control device, in which the control device calculates an oxygen saturation of the subject based on the detection signals from the red light sensor and the infrared light sensor, calculates, as an indicator value, an analysis result of time variation analysis on at least one of a red light signal intensity ratio indicating a ratio of an amplitude of a vibration component in the detection signal output by the red light sensor to a signal intensity of the detection signal and an infrared light signal intensity ratio indicating a ratio of an amplitude of a vibration component in the detection signal output by the infrared light sensor to a signal intensity of the detection signal, and displays the oxygen saturation and the analysis result on the display unit, and displays the oxygen saturation or the analysis result in different display formats when the analysis result is equal to or more than a predetermined threshold or when the analysis result is less than the threshold.

### BRIEF DESCRIPTION OF THE DRAWINGS

FIG. 1 is a schematic diagram showing a pulse oximeter according to an embodiment of the disclosure.

FIG. 2 is a schematic view showing a use state of the pulse oximeter according to the embodiment.

FIG. 3 is a block diagram showing a control device of the pulse oximeter according to the embodiment.

FIG. 4 shows an example of a detection signal.

FIG. 5 shows an absorption spectrum of oxygenated hemoglobin and reduced hemoglobin.

FIG. 6 shows an oxygen saturation data group, a red light signal intensity ratio, and an infrared light signal intensity ratio in the embodiment on the same time axis.

FIG. 7 shows a method for detecting a sudden peak point using a moving interval.

FIG. 8 shows the oxygen saturation data group, a coefficient of variation of the red light signal intensity ratio, and a coefficient of variation of the infrared light signal intensity ratio in the embodiment on the same time axis.

FIG. 9 shows abnormal value deletion processing performed by a data processing unit in the embodiment.

FIG. 10 shows interpolation processing performed by the data processing unit in the embodiment.

FIG. 11 is a schematic diagram showing an example of a display screen in the embodiment.

FIG. 12 is a schematic diagram showing another example of the display screen in the embodiment.

FIG. 13 is a flowchart showing an operation of the pulse oximeter in the embodiment.

FIG. 14 is a schematic diagram showing another embodiment of the disclosure.

### DESCRIPTION OF EMBODIMENTS

An embodiment of the disclosure will be described below.

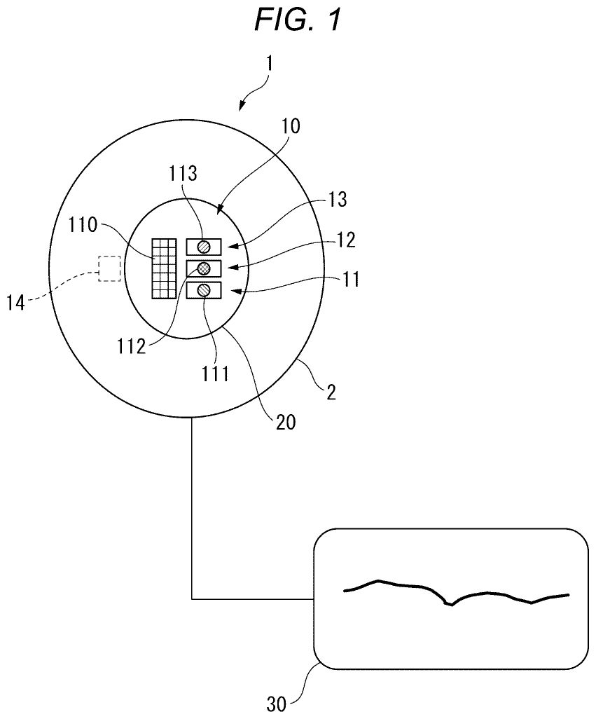

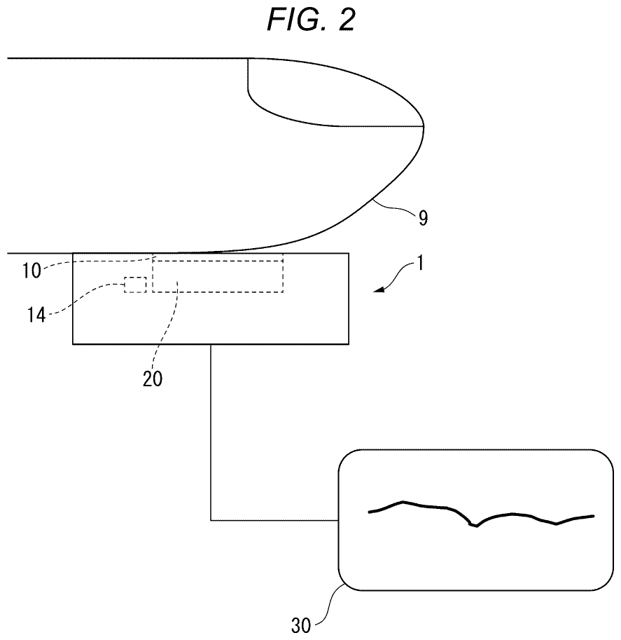

FIG. 1 is a schematic diagram showing a pulse oximeter according to the embodiment of the disclosure, and FIG. 2 is a schematic diagram showing a use state of the pulse oximeter according to the embodiment.

In FIGS. 1 and 2, a pulse oximeter 1 includes a sensor unit 10 and a control device 20. The sensor unit 10 and the control device 20 are accommodated in a case 2, and a display unit 30 is connected to the control device 20.

The sensor unit 10 is disposed on a surface of the case 2 and is held in close contact with a subject 9 such as a finger of a user during measurement.

A green light source 111, a red light source 112, an infrared light source 113, and an optical detector 110 are disposed in the sensor unit 10.

The green light source 111, the red light source 112, and the infrared light source 113 are, for example, light emitting diodes (LEDs) or laser diodes. It is desirable that an emission wavelength of the green light source 111 is 500 nm to 600 nm, an emission wavelength of the red light source 112 is 600 nm to 800 nm, and an emission wavelength of the infrared light source 113 is 800 nm to 1,000 nm.

The optical detector 110 is, for example, a silicon photodiode. Surfaces of the green light source 111, the red light source 112, the infrared light source 113, and the optical detector 110 of the sensor unit 10 are covered with a cover (not shown) made of a transparent resin-molded plate, such as acrylic or polycarbonate.

In the sensor unit 10, the green light source 111, the red light source 112, and the infrared light source 113 sequentially emit light under control of the control device 20, and light beams from the respective light sources are reflected by subcutaneous tissue of the subject 9 and returned to the optical detector 110. The control device 20 acquires detection signals from the optical detector 110 as a detection signal of green light, a detection signal of red light, and a detection signal of infrared light from a light emission timing of each light emitting element. The green light source 111 and the optical detector 110 constitute a green light sensor 11, the red light source 112 and the optical detector 110 constitute a red light sensor 12, and the infrared light source 113 and the optical detector 110 constitute an infrared light sensor 13.

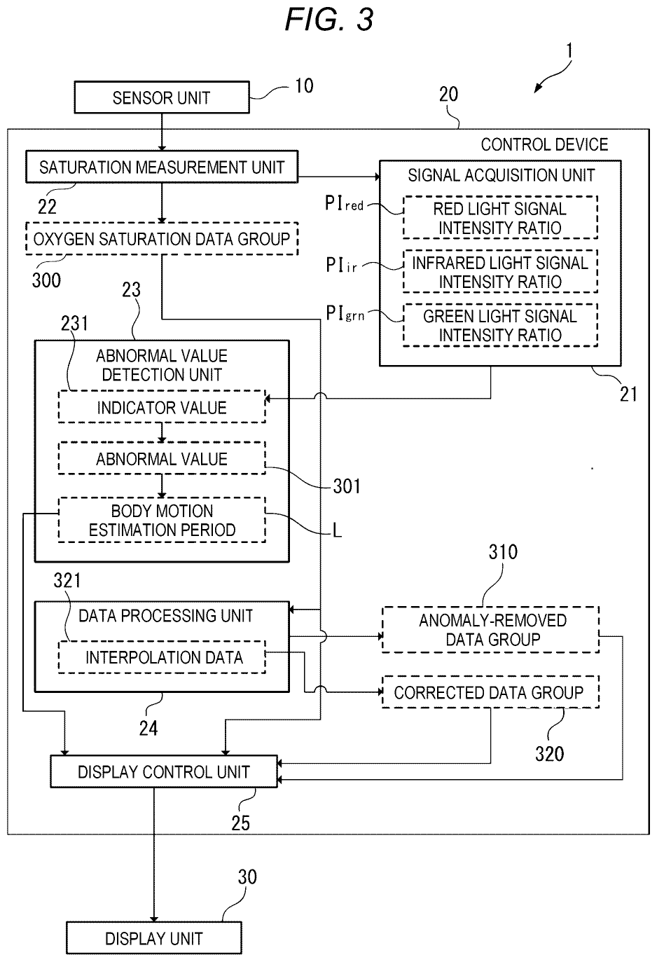

FIG. 3 is a block diagram showing a configuration of the control device 20 and a functional configuration of a processor.

The control device 20 is implemented by a small computer system and includes a memory that stores various data and a processor that implements a desired function (see FIG. 3) by executing a program stored in the memory.

In the case 2, a battery serving as a power supply of the sensor unit 10 and the control device 20 is stored, and an input and output terminal or the like of the control device 20 is provided (not shown).

The display unit 30 is connected to the control device 20 by wired or wireless signal unit, and can display a measurement result or the like from the control device 20. As the display unit 30, for example, in addition to a general-purpose image display panel, a portable information terminal such as a so-called smartphone can be used.

As shown in FIG. 3, the control device 20 functions as a signal acquisition unit 21, a saturation measurement unit 22, an abnormal value detection unit 23, a data processing unit 24, and a display control unit 25 by the processor executing a program.

The signal acquisition unit 21 controls the green light sensor 11, the red light sensor 12, and the infrared light sensor 13 of the sensor unit 10 connected to the control device 20, and acquires (receives) a detection signal.

The signal acquisition unit 21 calculates a signal intensity ratio based on the received detection signal. The signal intensity of the detection signal from each color sensor varies for light of each color. Therefore, the signal acquisition unit 21 calculates the signal intensity ratio based on the signal intensity of light of each color such that highly accurate processing is enabled in measurement of a blood oxygen saturation (SpO2) by the saturation measurement unit 22 to be described later and abnormal value detection by the abnormal value detection unit 23 to be described later. In the following description, an arterial blood oxygen saturation may be simply referred to as an oxygen saturation.

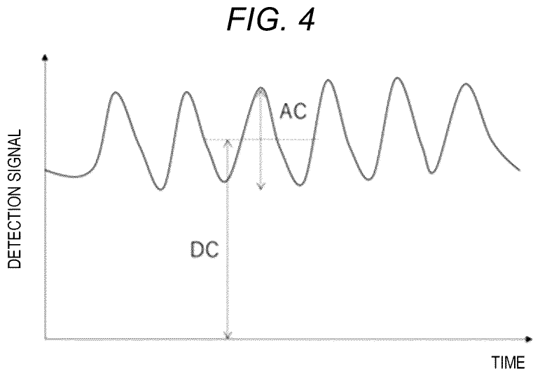

FIG. 4 shows an example of the detection signal.

For example, a pulse wave signal as shown in FIG. 4 is obtained by sampling the detection signal from one sensor at a predetermined sampling period. Here, an amplitude intensity of the detection signal (pulse wave signal) is AC, and a signal intensity is DC. The detection signal of light of each color is a pulse wave where a vibration component is superimposed on a DC component, the amplitude intensity AC is detected from an amplitude of the vibration component, and the DC intensity DC is detected at the center of the amplitude. When the amplitude intensity AC and the DC intensity DC are obtained, a pulse wave signal intensity ratio of the detection signal is obtained by a ratio of the amplitude intensity AC to the DC intensity DC (signal intensity ratio PI=AC/DC). Here, a signal intensity ratio related to red light (red light signal intensity ratio PI_{red}), a signal intensity ratio of infrared light (infrared light signal intensity ratio PIᵢᵣ), and a signal intensity ratio of green light (green light signal intensity ratio PI_{grn}) are obtained by the following formulas (1) to (3). AC_{red} is the amplitude intensity of the detection signal from the red light sensor 12, and DC_{red} is the signal intensity of the detection signal from the red light sensor 12. ACᵢᵣ is the amplitude intensity of the detection signal from the infrared light sensor 13, and DCᵢᵣ is the signal intensity of the detection signal from the infrared light sensor 13. AC_{grn} is the amplitude intensity of the detection signal from the green light sensor 11, and DC_{grn} is the signal intensity of the detection signal from the green light sensor 11.

$${PI}_{red} = \frac{{AC}_{red}}{{DC}_{red}} \qquad ( 1 )$$

$${PI}_{ir} = \frac{{AC}_{ir}}{{DC}_{ir}} \qquad ( 2 )$$

$${PI}_{grn} = \frac{{AC}_{grn}}{{DC}_{grn}} \qquad ( 3 )$$

The saturation measurement unit 22 calculates the arterial blood oxygen saturation of the subject 9 based on the red light signal intensity ratio PIred and the infrared light signal intensity ratio PIir, and records the arterial blood oxygen saturation in the memory as an oxygen saturation data group 300.

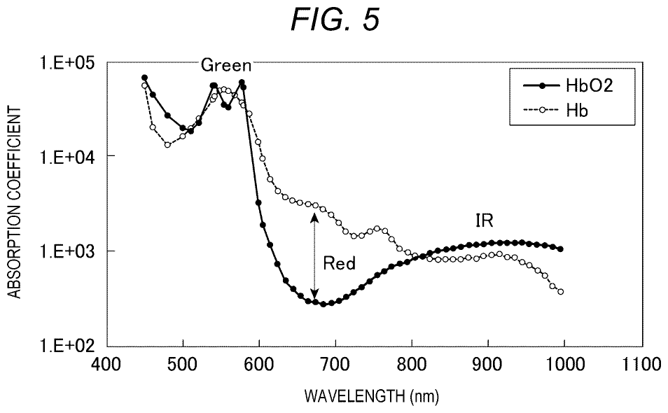

FIG. 5 shows an absorption spectrum of oxygenated hemoglobin and reduced hemoglobin.

Detection signals from the green light sensor 11, the red light sensor 12, and the infrared light sensor 13 are photoplethysmographic signals based on green light, red light, and infrared light transmitted through the subcutaneous tissue of the subject 9, and an absorption amount of light of each color into oxygenated hemoglobin and an absorption amount of light of each color into reduced hemoglobin are different. That is, an amount of light absorbed by both oxygenated hemoglobin and reduced hemoglobin is large for green light, and an amount of light absorbed by both oxygenated hemoglobin and reduced hemoglobin is relatively small for infrared light. Meanwhile, for red light, there is a large difference between an amount of light absorbed by oxygenated hemoglobin and an amount of light absorbed by reduced hemoglobin.

Therefore, the saturation measurement unit 22 can accurately measure the oxygen saturation based on a difference between the light absorption amounts of red light and infrared light based on the detection signals (photoplethysmographic signals) from the red light sensor 12 and the infrared light sensor 13. Specifically, a red-infrared signal ratio R between the red light signal intensity ratio PI_{red} calculated based on the red light detection signal and the infrared light signal intensity ratio PIᵢᵣ calculated based on the infrared light detection signal is calculated. In addition, mapping data indicating a relationship between the red-infrared signal ratio R and the oxygen saturation is stored in the memory in advance. Then, the saturation measurement unit 22 calculates the oxygen saturation based on the red-infrared signal ratio R and the mapping data, and calculates the oxygen saturation corresponding to each detection signal acquired at the predetermined sampling period to obtain the oxygen saturation data group 300.

FIG. 5 shows the absorption spectrum of oxygenated hemoglobin and reduced hemoglobin.

The detection signals from the green light sensor 11, the red light sensor 12, and the infrared light sensor 13 are photoplethysmographic signals based on green light, red light, and infrared light transmitted through the subcutaneous tissue of the subject 9, and the absorption amount of each color into oxygenated hemoglobin and the absorption amount of each color into reduced hemoglobin are different. That is, the amount of light absorbed by both oxygenated hemoglobin and reduced hemoglobin is large for green light, and the amount of light absorbed by both oxygenated hemoglobin and reduced hemoglobin is relatively small for infrared light. Meanwhile, for red light, there is a large difference between the amount of light absorbed by oxygenated hemoglobin and the amount of light absorbed by reduced hemoglobin.

Therefore, the saturation measurement unit 22 can accurately measure the oxygen saturation based on the difference between the light absorption amounts of red light and infrared light based on the detection signals (photoplethysmographic signals) from the red light sensor 12 and the infrared light sensor 13. Specifically, the red-infrared signal ratio R between the red light detection signal and the infrared light detection signal is calculated. In addition, the mapping data indicating the relationship between the red-infrared signal ratio R and the oxygen saturation is stored in the memory in advance. Then, the saturation measurement unit 22 calculates the oxygen saturation based on the red-infrared signal ratio R and the mapping data, and calculates the oxygen saturation corresponding to each detection signal acquired at the predetermined sampling period to obtain the oxygen saturation data group 300.

The saturation measurement unit 22 obtains the red-infrared signal ratio R by the following formula (4) using the red light signal intensity ratio PI_{red} and the infrared light signal intensity ratio PIᵢᵣ.

$$R = \frac{{PI}_{red}}{{PI}_{ir}} = \frac{{AC}_{red} / {DC}_{red}}{{AC}_{ir} / {DC}_{ir}} \qquad ( 4 )$$

The abnormal value detection unit 23 detects a body motion artifact (abnormal value 301) caused by a body motion of the subject 9, which appears in the oxygen saturation data group 300.

The abnormal value detection unit 23 detects the body motion artifact using an indicator value 231 based on at least one of the red light signal intensity ratio PI_{red} and the infrared light signal intensity ratio PIᵢᵣ obtained by the above formulas (1) and (2) as detection of the body motion artifact.

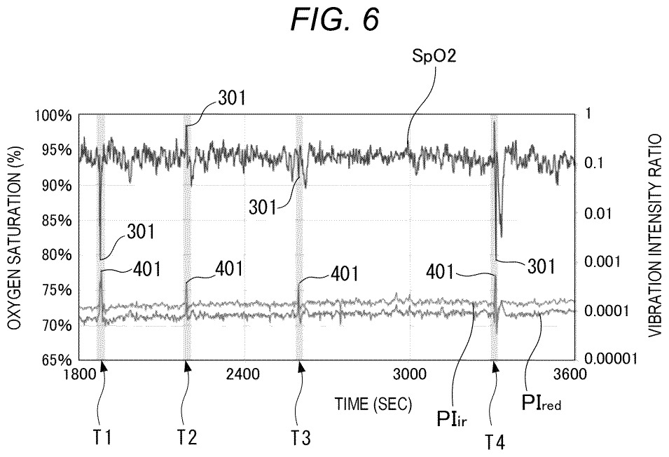

FIG. 6 shows the oxygen saturation data group 300, the red light signal intensity ratio PI_{red}, and the infrared light signal intensity ratio PIᵢᵣ on the same time axis. That is, a curve indicating the red light signal intensity ratio PI_{red} in FIG. 6 is a data group of the red light signal intensity ratio PI_{red} indicating a change over time in the red light signal intensity ratio PI_{red} calculated based on the detection signal obtained from the red light sensor 12 acquired at the predetermined sampling period. A curve indicating the infrared light signal intensity ratio PIᵢᵣ in FIG. 6 is a data group of the infrared light signal intensity ratio PIᵢᵣ indicating a change over time in the infrared light signal intensity ratio PIᵢᵣ calculated based on the detection signal obtained from the infrared light sensor 13 acquired at the predetermined sampling period.

In FIG. 6, at times T1 to T4, it can be seen that at least one of the red light signal intensity ratio PI_{red} and the infrared light signal intensity ratio PIᵢᵣ has a large peak value variation as compared to the red light signal intensity ratio PIr_{ed} and the infrared light signal intensity ratio PIᵢᵣ in other time periods. Changes in the red light signal intensity ratio PI_{red} and the infrared light signal intensity ratio PIᵢᵣ at these times T1 to T4 are not due to sudden blood flow changes, but are disturbances in the detection signal due to the body motion. In particular, in the reflective pulse oximeter 1 that emits red light, infrared light, and green light from the sensor unit 10 and receives light reflected in a body of the subject 9, the red light signal intensity ratio PI_{red} and the infrared light signal intensity ratio PIᵢᵣ are very small. Therefore, even slight movement such as turning over in sleep of the subject 9 may cause a sudden peak point (artifact) to be mixed into the red light signal intensity ratio PI_{red} or the infrared light signal intensity ratio PIᵢᵣ. Although the green light signal intensity ratio PI_{grn} is not shown in FIG. 6, the same applies to the green light signal intensity ratio PI_{grn}.

Further, as shown in FIG. 6, when there is a sudden peak point 401 in the red light signal intensity ratio PI_{red} and the infrared light signal intensity ratio PIᵢᵣ, the oxygen saturation also suddenly fluctuates. This is because the blood oxygen saturation is not changed but a sudden body motion artifact is mixed.

In the disclosure, the sudden peak point 401 refers to a peak point among a plurality of peak points of the red light signal intensity ratio PI_{red} or the infrared light signal intensity ratio PIᵢᵣ within a predetermined period, where a difference from a peak value of another peak point is equal to or more than a predetermined value and the number of such peak points in the predetermined period is equal to or less than a predetermined number set in advance. For example, in the example in FIG. 6, a time (for example, 180 seconds) required to determine sleep apnea syndrome is set as the predetermined period, and when there are two or less peak points at which the peak value is larger than other peak points by a predetermined value or more in the predetermined period, a peak point at which the peak value is larger than other peak points by the predetermined value or more is treated as the sudden peak point.

Examples of a method for detecting the body motion artifact by the abnormal value detection unit 23 include the following two methods.

A first method is a method of calculating the sudden peak point 401 using, as the indicator value 231, at least one signal intensity ratio PI (the red light signal intensity ratio PI_{red}, the infrared light signal intensity ratio PIᵢᵣ, and the green light signal intensity ratio PI_{grn}) calculated based on the detection signals of the respective colors acquired at the predetermined sampling period. In this method, the abnormal value detection unit 23 calculates a second derivative curve of the signal intensity ratio PI (the red light signal intensity ratio PI_{red}, the infrared light signal intensity ratio PIᵢᵣ, and the green light signal intensity ratio PI_{grn}) and calculates a zero-crossing point thereof. Accordingly, the peak point in the signal intensity ratio PI can be detected, and the peak value of each peak point can be calculated. Therefore, an average peak value of peak points in the predetermined period is calculated, and a peak point having a peak value larger than the average peak value by a predetermined value or more can be detected as the sudden peak point 401.

Alternatively, the sudden peak point 401 may be detected by setting a predetermined period (hereinafter, referred to as a moving interval in the disclosure) in the signal intensity ratio PI (the red light signal intensity ratio PI_{red}, the infrared light signal intensity ratio PIᵢᵣ, and the green light signal intensity ratio PI_{grn}) and detecting a maximum value of the signal intensity ratio PI in the moving interval.

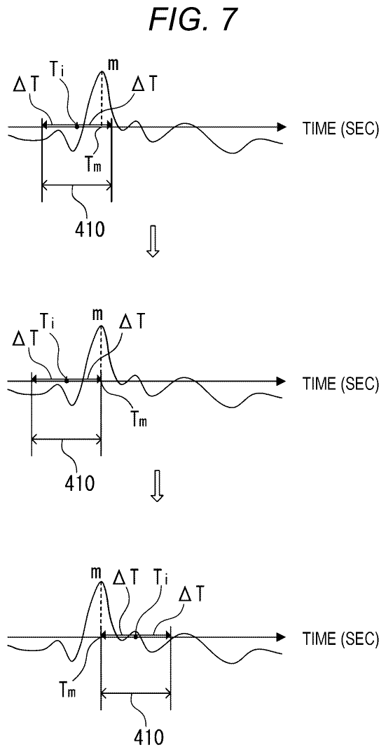

FIG. 7 shows a method for detecting the sudden peak point 401 using the moving interval.

The abnormal value detection unit 23 sets a certain measurement time as a target time T₁, sets an analysis range of ΔT (sec) before and after the target time T1 as a moving interval 410, and calculates a maximum value m of the signal intensity ratio PI in the moving interval 410 and a time Tm at which the maximum value m is obtained. When the maximum value m and the time Tm at which the maximum value m is obtained do not change in a predetermined range when the target time T1 is sequentially advanced (the target time T1 is changed to move the moving interval), the abnormal value detection unit 23 treats the maximum value m as the sudden peak point 401. The predetermined range is, for example, (target time Tᵢ)±(time length of analysis range 2×ΔT). Alternatively, the interval may be an interval from (Tm−ΔT)±ΔT to (Tm+AT)±ΔT.

In the first method, the abnormal value detection unit 23 detects data (oxygen saturation) of the oxygen saturation data group 300 corresponding to the time Tm when the detected sudden peak point 401 is obtained as the abnormal value 301. At this time, it is preferable that the abnormal value detection unit 23 detects the abnormal value 301 corresponding to the peak point 401 when the sudden peak point 401 is obtained using any one of the red light signal intensity ratio PI_{red}, the infrared light signal intensity ratio PIᵢᵣ, and the green light signal intensity ratio PI_{grn} as the indicator value 231. The abnormal value detection unit 23 may detect the abnormal value corresponding to the peak point 401 when the sudden peak point 401 is obtained at the same position in any two or all of the signal intensity ratios PI among the red light signal intensity ratio PI_{red}, the infrared light signal intensity ratio PIᵢᵣ, and the green light signal intensity ratio PI_{grn}.

A second method is a method of using an analysis result of time variation analysis on the signal intensity ratios PI (the red light signal intensity ratio PI_{red}, the infrared light signal intensity ratio PIᵢᵣ, and the green light signal intensity ratio PI_{grn}) as the indicator value 231.

As the time variation analysis, for example, an analysis range of Δt (sec) before and after the predetermined target time T1 is set, and a variance v or a standard deviation s of the signal intensity ratio PI at n points within the analysis range, or a coefficient of variation cv for the signal intensity ratio PI is calculated as shown in the following formulas (5) to (7). Here, At (sec) for setting the analysis range may be the same time length as the moving interval 410, and in this case, Δt=ΔT.

$$v = \frac{1}{n} {\sum }_{i = 1}^{n} {( {x}_{i} - \overline{x} )}^{2} \qquad ( 5 )$$

$$s = \sqrt{v} = \sqrt{\frac{1}{n} {\sum }_{i = 1}^{n} {( {x}_{i} - \overline{x} )}^{2}} \qquad ( 6 )$$

$$cv = \frac{s}{\overline{x}} = \frac{\sqrt{\frac{1}{n} {\sum }_{i = 1}^{n} {( {x}_{i} - \overline{x} )}^{2}}}{\overline{x}} \qquad ( 7 )$$

In formulas (5) to (7), xᵢ is the signal intensity ratio PI (the red light signal intensity ratio PI_{red}, the infrared light signal intensity ratio PIᵢᵣ, and the green light signal intensity ratio PI_{grn}) within the analysis range, and x⁻ is an average value of the signal intensity ratio PI within the analysis range.

When the second method is used, the abnormal value detection unit 23 calculates any one of the variance v, the standard deviation s, and the coefficient of variation cv for the signal intensity ratio PI, sets any one of the variance v, the standard deviation s, and the coefficient of variation cv thus calculated as the indicator value 231, and determines whether the indicator value 231 exceeds a predetermined threshold. In the analysis range, any one of the variance v, the standard deviation s, and the coefficient of variation cv exceeding the predetermined threshold means that a variation in the signal intensity ratio PI within the analysis range is large, and indicates that the sudden peak point 401 is present. The threshold when the variance v is used, the threshold when the standard deviation s is used, and the threshold when the coefficient of variation cv is used are different from each other.

In particular, the coefficient of variation cv is a value obtained by normalizing the standard deviation s by an average value as shown in formula (7), and can be used as a consistent detection indicator regardless of a difference between devices of the individual pulse oximeters 1 or a difference between measurement sites, which is preferable.

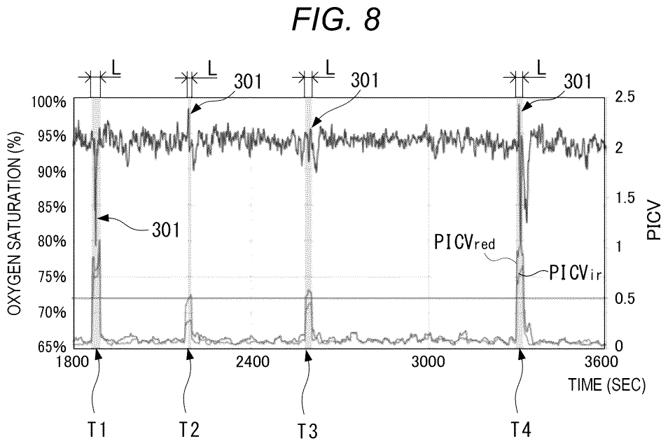

FIG. 8 shows the oxygen saturation data group 300, a coefficient of variation PICV_{red} of the red light signal intensity ratio PI_{red}, and a coefficient of variation PICVᵢᵣ of the infrared light signal intensity ratio PIᵢᵣ on the same time axis.

When the second method is used, as shown in FIG. 8, the body motion artifact can still be detected at the same time positions (T1 to T4) as in FIG. 6.

Although a coefficient of variation PICV_{grn} of the green light signal intensity ratio PI_{grn} is not shown in FIG. 8, the same applies to the coefficient of variation PICV_{grn} of the green light signal intensity ratio PI_{grn}.

In the second method, the abnormal value detection unit 23 detects, as the abnormal value 301, data (oxygen saturation) of the oxygen saturation data group 300 corresponding to a range where the analysis result as the indicator value 231 exceeds the threshold.

At this time, similarly to the first method, the abnormal value detection unit 23 may determine that there is the abnormal value 301 when any of analysis results of the time variation analysis on the red light signal intensity ratio PI_{red}, the infrared light signal intensity ratio PIᵢᵣ, and the green light signal intensity ratio PI_{grn} exceeds the threshold. Alternatively, the abnormal value detection unit 23 may determine that there is the abnormal value when any two or all of the analysis results of the time variation analysis on the red light signal intensity ratio PI_{red}, the infrared light signal intensity ratio PIᵢᵣ, and the green light signal intensity ratio PI_{grn} exceed the threshold.

Further, the abnormal value detection unit 23 estimates a period (body motion period L) in which the subject 9 has a body motion.

For example, when the abnormal value 301 is detected by the first method, the oxygen saturation at one point corresponding to the sudden peak point 401 is detected as the abnormal value 301. In this case, the abnormal value detection unit 23 estimates the body motion period L including a predetermined time OT before and after the time T1 when the abnormal value 301 is detected. Alternatively, the data processing unit 24 may set, as the body motion period L, a range where the oxygen saturation is equal to or less than a predetermined value, including the time T₁ when the abnormal value 301 is detected.

Meanwhile, in the second method, the analysis result of the time variation analysis indicating whether the sudden peak point 401 is within the analysis range (whether the abnormal value 301 is present in the analysis range) is calculated. Therefore, when the sudden peak point is within the analysis range, as shown in FIG. 8, the analysis result exceeds the threshold within a range having a predetermined time length. In this case, the abnormal value detection unit 23 may estimate the entire range where the analysis result exceeds the threshold as the body motion period L.

The data processing unit 24 generates an anomaly-removed data group 310 by deleting, from the oxygen saturation data group 300, the abnormal value 301 (oxygen saturation) detected by the abnormal value detection unit 23 and data (oxygen saturation) in the body motion period L.

The data processing unit 24 further interpolates a deleted portion with interpolation data 321 to create a corrected data group 320. Any data interpolation method such as linear interpolation or spline interpolation can be used for data interpolation processing.

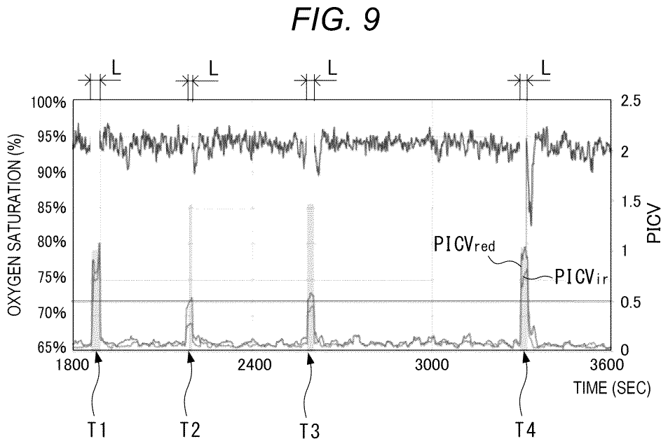

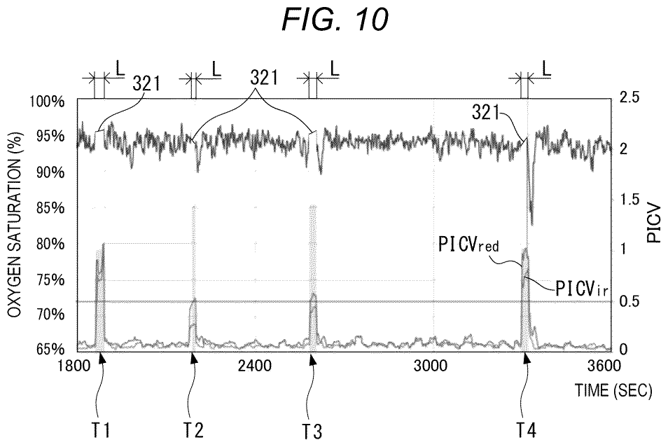

FIGS. 9 and 10 show the processing of deleting and interpolating the abnormal value 301 by the data processing unit 24.

For example, in FIG. 9, the data processing unit 24 deletes data of the interval (body motion period L) in the oxygen saturation data group 300 based on the abnormal value detected by the abnormal value detection unit 23 to obtain the anomaly-removed data group 310.

In FIG. 10, the data processing unit 24 interpolates the interval where the data is deleted in the oxygen saturation data group 300 with the interpolation data 321, and treats the oxygen saturation data group 300 interpolated with interpolation data 321 as the corrected data group 320.

The display control unit 25 displays, on the display unit 30, various data calculated by the control device 20.

Examples of display contents include the oxygen saturation data group 300, the anomaly-removed data group 310, the corrected data group 320, and the indicator value 231 for detecting the abnormal value 301. In addition, an alert display based on the oxygen saturation data group 300, the indicator value 231, or the like, the body motion period L estimated based on the abnormal value 301, or the like may be displayed. Further, an interval where the oxygen saturation decreases over a predetermined time or the number of times the oxygen saturation decreases may be calculated based on the indicator value 231, and a calculation result may be displayed on the display unit 30.

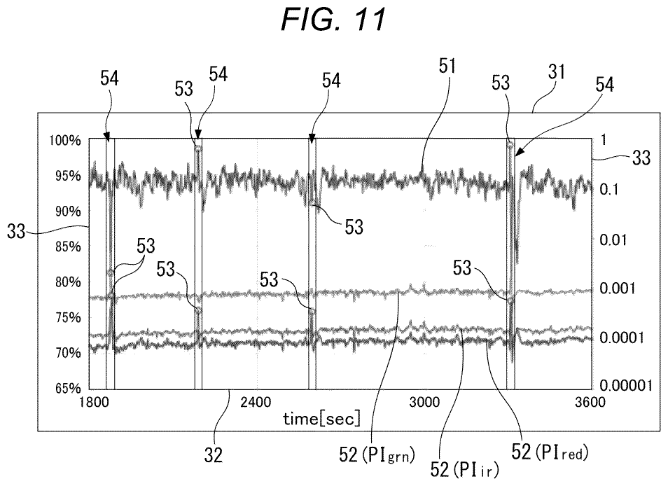

FIG. 11 shows an example of a display screen 31 displayed on the display unit 30 according to the embodiment.

For example, the display screen 31 shown in FIG. 11 includes an oxygen saturation graph 51, an indicator value graph 52, a peak point image 53, and a body motion display unit 54 along a time axis 32 displayed at a lower portion.

The oxygen saturation graph 51 is a graph showing the oxygen saturation data group 300 calculated by the saturation measurement unit 22, the anomaly-removed data group 310 calculated by the data processing unit 24, or the corrected data group 320. A switching icon or the like through which the data group displayed in the oxygen saturation graph 51 can be switched may be displayed on the display screen 31. In this case, when the switching icon is selected by the user, the display control unit 25 switches and displays the oxygen saturation data group 300, the anomaly-removed data group 310, and the corrected data group 320.

FIG. 11 is an example in which the oxygen saturation data group 300 is displayed as the oxygen saturation graph 51.

The indicator value graph 52 is a graph showing the indicator value 231 used when the abnormal value is detected by the abnormal value detection unit 23. In the example in FIG. 11, the abnormal value is detected by the first method, and the red light signal intensity ratio PI_{red} and the infrared light signal intensity ratio PIᵢᵣ are displayed in the indicator value graph 52 as the indicator value 231. The indicator value graph 52 is displayed in a display format different from that of the oxygen saturation graph 51 so as to be distinguishable from the oxygen saturation graph 51, and is displayed with a color, a line type, or a line width different from that of the oxygen saturation graph 51, for example.

The peak point image 53 indicates the sudden peak point 401 in the indicator value and the abnormal value of the oxygen saturation corresponding to the peak point 401, which are detected by the abnormal value detection unit 23 using the first method. For example, the peak point image 53 is preferably displayed with a color or a line type different from that of the oxygen saturation graph 51 or the indicator value graph 52 such that a position in the oxygen saturation graph 51 or the indicator value graph 52 can be clearly understood. For example, in the example in FIG. 11, a circular image is superimposed on the oxygen saturation graph 51 or the indicator value graph 52 and displayed as the peak point image 53.

The body motion display unit 54 indicates a range of the body motion period L detected by the abnormal value detection unit 23. The body motion display unit 54 is displayed in a display format different from that of the oxygen saturation graph 51, the indicator value graph 52, and the peak point image 53 such that a period in which the body motion is estimated to have occurred on the time axis 32 can be understood.

In addition, on both sides of the display screen 31, vertical axis displays 33 indicating numerical values of the oxygen saturation graph 51 and the indicator value graph 52 are displayed.

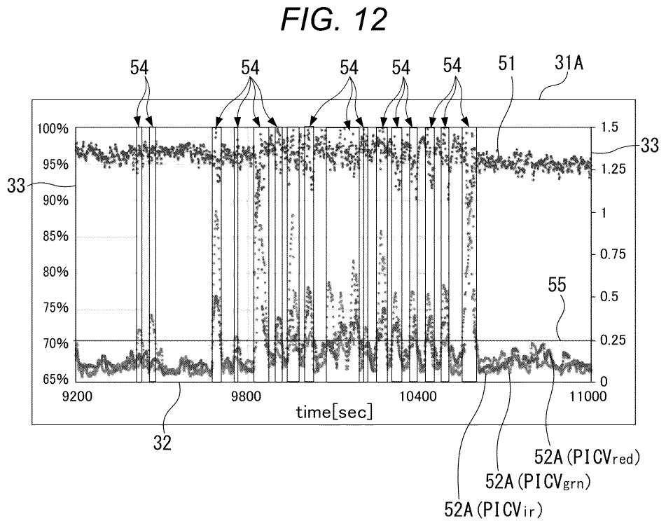

FIG. 12 shows another example of a display screen 31A displayed on the display unit 30 of the embodiment.

FIG. 11 described above is an example of the display screen 31 when the abnormal value detection unit 23 detects the abnormal value 301 using the first method. Meanwhile, FIG. 12 is an example of the display screen 31A when the abnormal value detection unit 23 detects the abnormal value 301 using the second method.

For example, the display screen 31A shown in FIG. 12 includes the oxygen saturation graph 51, an indicator value graph 52A, a threshold display unit 55, and the body motion display unit 54 along the time axis 32 displayed at a lower portion. In addition, on both sides of the display screen 31A, the vertical axis displays 33 indicating the numerical values of the oxygen saturation graph 51 and the indicator value graph 52 are displayed as in FIG. 11.

The oxygen saturation graph 51 is similar to the display screen 31 shown in FIG. 11, and is a graph showing the oxygen saturation data group 300, the anomaly-removed data group 310, or the corrected data group 320. As in the case of FIG. 11, a switching icon or the like through which the data group displayed in the oxygen saturation graph 51 can be switched may be displayed on the display screen 31.

FIG. 12 is an example in which the anomaly-removed data group 310 is displayed as the oxygen saturation graph 51.

The indicator value graph 52A is a graph showing the indicator value 231 used when the abnormal value is detected by the abnormal value detection unit 23. As described above, the example in FIG. 12 is the display screen 31A when the abnormal value 301 is detected by the second method, and the analysis result of the time variation analysis on the signal intensity ratio PI (the red light signal intensity ratio PI_{red}, the infrared light signal intensity ratio PIᵢᵣ, and the green light signal intensity ratio PI_{grn}) as the indicator value 231 is displayed as the indicator value graph 52A. For example, in FIG. 12, the coefficient of variation cv of the signal intensity ratio PI is used as the analysis result, and the coefficient of variation PICV_{red} of the red light signal intensity ratio PI_{red}, the coefficient of variation PICVᵢᵣ of the infrared light signal intensity ratio PIᵢᵣ, and the coefficient of variation PICV_{grn} of the green light signal intensity ratio PI_{grn} are displayed. The indicator value graph 52A is displayed in a display format different from that of the oxygen saturation graph 51 so as to be distinguishable from the oxygen saturation graph 51, and is displayed with a color, a line type, or a line width different from that of the oxygen saturation graph 51, for example.

The threshold display unit 55 indicates a threshold of the analysis result (in this example, the coefficient of variation cv) of the time variation analysis by the abnormal value detection unit 23. The threshold display unit 55 is not limited to a horizontal straight line as shown in FIG. 12, and may be displayed by another mark or shape.

The body motion display unit 54 is similar to that in FIG. 11 and indicates the range of the body motion period L detected by the abnormal value detection unit 23.

### Operation of Pulse Oximeter

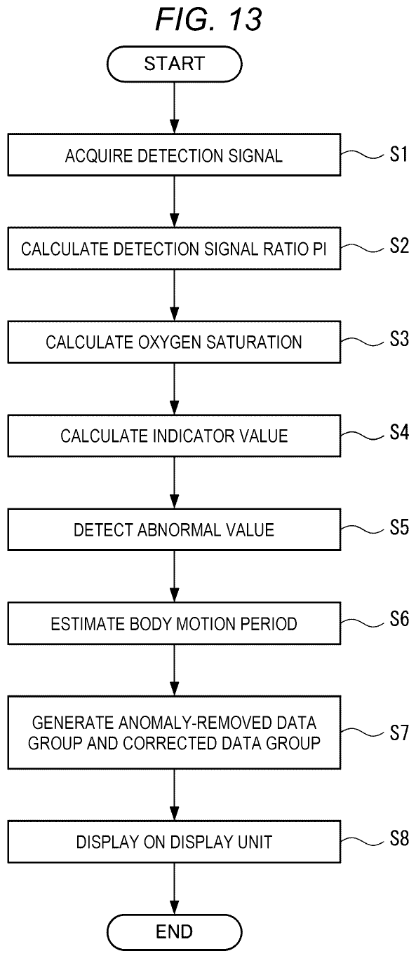

FIG. 13 shows an overview of processing executed by the pulse oximeter 1 of the embodiment.

In the pulse oximeter 1, when the processor of the control device 20 executes a program, the following processing is executed by each unit of the sensor unit 10 and the control device 20.

First, the signal acquisition unit 21 controls the sensor unit 10 to irradiate the subject 9 with green light, red light, and infrared light and detect the green light, the red light, and the infrared light transmitted through or reflected from the subject 9. Accordingly, the signal acquisition unit 21 acquires the detection signal of each color from the sensor unit 10 (step S1).

The signal acquisition unit 21 calculates the signal intensity ratio PI (the red light signal intensity ratio PI_{red}, the infrared light signal intensity ratio PIᵢᵣ, and the green light signal intensity ratio PI_{grn}) of light of each color based on the acquired detection signal of light of each color (step S2).

Then, the saturation measurement unit 22 measures the oxygen saturation of the subject 9 using the red light signal intensity ratio PI_{red}, the infrared light signal intensity ratio PIᵢᵣ, and the red-infrared signal ratio R recorded in the memory, and records the oxygen saturation as the oxygen saturation data group 300 (step S3).

Next, the abnormal value detection unit 23 calculates the indicator value 231 for detecting the abnormal value (step S4). Here, when the abnormal value detection unit 23 detects the abnormal value by the first method, the signal intensity ratio PI (PI_{red}, PIᵢᵣ, and PI_{grn}) of light of each color is calculated as the indicator value 231 based on the detection signal of each color. When the abnormal value detection unit 23 detects the abnormal value by the second method, the analysis result (for example, the coefficient of variation cv (PICV_{red}, PICVᵢᵣ, and PICV_{grn})) of the time variation analysis on the signal intensity ratio PI (PI_{red}, PIᵢᵣ, and PI_{grn}) of light of each color is calculated as the indicator value 231.

The method for detecting the abnormal value by the abnormal value detection unit 23 may be appropriately selected by the user, and the indicator value 231 may be calculated according to selection of the user.

Next, the abnormal value detection unit 23 detects the abnormal value 301 appearing in the oxygen saturation data group 300 based on the calculated indicator value 231 (step S5). The method for detecting the abnormal value 301 is as described above, and the first method or the second method is used. As described above, the user may be allowed to select either the first method or the second method.

In step S5, the abnormal value detection unit 23 estimates the body motion period L based on the abnormal value 301 (step S6).

Next, the data processing unit 24 removes the abnormal value 301 and the data in the body motion period L from the oxygen saturation data group 300 to generate the anomaly-removed data group 310, and further performs interpolation processing for the removed data to generate the interpolation data 321 and generate the corrected data group 320 (step S7).

Thereafter, the display control unit 25 displays, on the display unit 30, the oxygen saturation graph 51 based on the oxygen saturation data group 300, the anomaly-removed data group 310, or the corrected data group 320, the indicator value graph 52 based on the indicator value, and the like on the display unit 30 as shown in FIGS. 11 and 12 (step S8).

In the pulse oximeter 1 of the embodiment, it is possible to check whether a function is normal by the following operation.

A film or sheet that can prevent transmission of light having an inspection wavelength of the green light sensor 11, the red light sensor 12, and the infrared light sensor 13 is prepared, and measurement is performed in a state where the film or sheet is interposed between the subject 9 and the sensor unit 10 of the pulse oximeter 1.

When the pulse oximeter 1 detects the abnormal value 301 using the analysis result (the variance v, the standard deviation s, and the coefficient of variation cv) of the time variation analysis on the red light signal intensity ratio PI_{red} or the red light signal intensity ratio PI_{red}, a function of detecting the body motion artifact is impaired when the film preventing the red light is interposed, and the function is recovered by removing the film.

When the pulse oximeter 1 detects the abnormal value 301 using the analysis result of the time variation analysis on the infrared light signal intensity ratio PIᵢᵣ or the infrared light signal intensity ratio PIᵢᵣ, the function of detecting the body motion artifact is impaired when the film preventing the infrared light is interposed, and the function is recovered by removing the film.

When the pulse oximeter 1 detects the abnormal value 301 using the analysis result of the time variation analysis on the green light signal intensity ratio PI_{grn} or the green light signal intensity ratio PI_{grn}, the function of detecting the body motion artifact is impaired when the film preventing the green light is interposed, and the function is recovered by removing the film.

In this way, the function of the pulse oximeter 1 can be determined by blocking the inspection light.

### Functions and Effects of Embodiment

The pulse oximeter 1 of the embodiment includes the red light sensor 12 that irradiates the subject 9 with red light and detects the red light transmitted through or reflected from the subject 9, the infrared light sensor 13 that irradiates the subject 9 with infrared light and detects the infrared light transmitted through or reflected from the subject 9, and the control device 20 that processes the detection signals from the red light sensor 12 and the infrared light sensor 13.

The control device 20 includes the saturation measurement unit 22 that measures the oxygen saturation of the subject 9 based on the detection signals from the red light sensor 12 and the infrared light sensor 13 and records the oxygen saturation as the oxygen saturation data group 300, and the abnormal value detection unit 23 that detects the abnormal value 301 caused by the body motion of the subject 9 appearing in the oxygen saturation data group 300 based on the signal intensity ratio PI.

In such a pulse oximeter, the abnormal value 301 of the oxygen saturation based on the body motion of the subject 9 can be detected using at least one of the red light signal intensity ratio PI_{red} and the infrared light signal intensity ratio PIᵢᵣ for calculating at least the oxygen saturation, and oxygen saturation measurement processing can be performed with high accuracy by excluding the abnormal value 301.

For example, the abnormal value 301 based on the body motion can be detected without using another sensor such as an acceleration sensor. That is, since the abnormal value 301 based on the body motion is detected using the detection signal from the red light sensor 12 or the infrared light sensor 13 for measuring the oxygen saturation, it is not required to provide another configuration for detecting the body motion such as an acceleration sensor, and thus the configuration of the pulse oximeter 1 can be simplified. In addition, when the acceleration sensor that directly detects a physical motion of the subject 9 is used, there may be cases where a minute motion of the subject 9 cannot be detected, and in the case where the body motion is detected using the indicator value 231 based on at least one signal intensity ratio PI calculated based on the detection signal of light of each color, the abnormal value 301 due to the minute body motion of the subject 9 can also be accurately detected.

In the pulse oximeter 1 of the embodiment, when the abnormal value detection unit 23 detects the abnormal value 301 using the first method, the abnormal value detection unit 23 detects, as the abnormal value 301, the oxygen saturation at the timing when the sudden peak point 401 is obtained when the sudden peak point 401 whose difference from another peak value within the predetermined period is equal to or more than the predetermined value is detected among the plurality of peak values in at least one signal intensity ratio PI calculated based on the detection signal of light of each color.

When the body motion of the subject 9 occurs, a disturbance enters the detection signal, and as a result, the sudden peak point 401 appears in any signal intensity ratio PI calculated based on the detection signal of light of each color. In the embodiment, when the abnormal value detection unit 23 detects the abnormal value 301 by the first method, such a sudden peak point 401 appearing in at least one signal intensity ratio PI calculated based on the detection signal of light of each color is detected. Accordingly, the oxygen saturation at the timing when the sudden peak point 401 is detected can be easily detected as the abnormal value 301 based on the body motion.

In the pulse oximeter 1 of the embodiment, when the abnormal value detection unit 23 detects the abnormal value 301 by the second method, the abnormal value detection unit 23 may detect the abnormal value 301 in the oxygen saturation data group 300 by the time variation analysis on the signal intensity ratio PI (the red light signal intensity ratio PI_{red}, the infrared light signal intensity ratio PIᵢᵣ, and the green light signal intensity ratio PI_{grn}) within the time range Δt [sec] before and after the predetermined target time Tᵢ.

When the body motion of the subject 9 occurs, a disturbance enters the detection signal, and thus the sudden peak point 401 appears in the indicator value 231 as described above. By performing the time variation analysis on the signal intensity ratio PI, the occurrence of such a sudden peak point 401 can be easily detected, and accordingly, the abnormal value 301 based on the body motion can be easily detected.

In the pulse oximeter 1 of the embodiment, as the time variation analysis, the abnormal value detection unit 23 calculates the standard deviation s or the variance v of the signal intensity ratio PI at the n points based on the detection signals at the n points output within the predetermined time range, and detects the abnormal value 301 based on the standard deviation s or the variance v.

In a variation of the signal intensity ratio PI in the predetermined period, when the variance v or the standard deviation s increases, it means that the sudden peak point 401 is present in the predetermined period, and thus presence or absence of the sudden peak point 401 can be accurately detected by the variance v or the standard deviation s.

In the pulse oximeter 1 of the embodiment, it is more preferable that the abnormal value detection unit 23 calculates the coefficient of variation cv obtained by normalizing the standard deviation s by the average value of the signal intensity ratio PI as the time variation analysis, and detects, as the abnormal value 301, the oxygen saturation at a predetermined timing when the coefficient of variation cv is equal to or more than the predetermined threshold.

Accordingly, it is possible to detect the abnormal value with high accuracy regardless of a difference between devices of the individual pulse oximeters 1 or a difference between measurement sites.

The pulse oximeter 1 of the embodiment includes the sensor unit 10 including the red light sensor 12 and the infrared light sensor 13, the control device 20 that processes the detection signal of each light sensor, and the display unit 30 that displays information obtained by the control device 20. Then, the control device 20 generates the oxygen saturation data group 300 indicating the oxygen saturation of the subject 9 based on the detection signals of the red light sensor 12 and the infrared light sensor 13, calculates the indicator value 231 based on at least one of the red light signal intensity ratio PI_{red} and the infrared light signal intensity ratio PIᵢᵣ, and displays a graph in which the oxygen saturation data group and the indicator value 231 are displayed on the same time axis 32 on the display unit 30.

Accordingly, the indicator value 231 for detecting the abnormal value 301 based on the body motion can be displayed on the display unit 30 together with the oxygen saturation data group 300, and the user can easily grasp a time when the body motion of the subject 9 occurs based on the indicator value 231.

In the pulse oximeter 1 of the embodiment, the display control unit 25 of the control device 20 estimates the body motion period L based on the indicator value 231 and displays the body motion period L superimposedly on the graph.

Accordingly, even when the user has no knowledge about the indicator value 231, the body motion of the subject 9 can be easily checked based on the body motion period L displayed on the graph.

In the pulse oximeter 1 of the embodiment, when the abnormal value 301 is detected by the first method, the display control unit 25 of the control device 20 displays, superimposedly on the graph, the peak point image 53 indicating the sudden peak point 401 in the signal intensity ratio PI that is the indicator value 231.

Accordingly, the user can easily grasp the time when the body motion of the subject 9 occurs and the indicator value 231 at that time.

In the pulse oximeter 1 of the embodiment, when the abnormal value 301 is detected by the second method, the display control unit 25 of the control device 20 calculates the analysis result of the time variation analysis on the signal intensity ratio PI as the indicator value 231 and displays the analysis result as the indicator value graph 52 in the graph.

Accordingly, as in the above disclosure, the user can grasp the time when the body motion of the subject 9 occurs.

### Other Embodiments

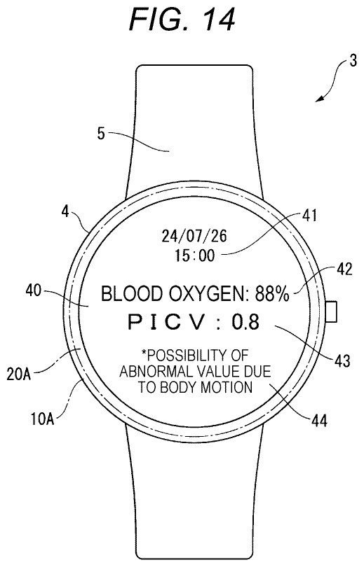

FIG. 14 shows another embodiment of the disclosure.

In FIG. 14, a pulse oximeter 3 has a flat disk-shaped case 4 and a belt 5, and can be worn on a wrist of the user.

A display unit 40 is formed at a surface of the case 4, and a current time display 41, an oxygen saturation display 42, a body motion indicator display 43, and an alert display 44 are displayed on the display unit 40. A control device 20A is stored inside the case 4, and a sensor unit 10A is formed at a back side of the case 4.

The sensor unit 10A includes the red light sensor 12 and the infrared light sensor 13 similarly to the sensor unit 10 of the embodiment described above. The red light sensor 12 and the infrared light sensor 13 are disposed to be in close contact with a surface of the wrist of the user when the pulse oximeter 3 is attached to the wrist of the user with the belt 5, and the surface of the wrist serves as the subject 9.

The control device 20A is implemented similarly to the control device 20 described above, measures the oxygen saturation of the subject 9 based on the detection signals from the red light sensor 12 and the infrared light sensor 13 to generate the oxygen saturation data group 300, and calculates the indicator value 231 for detecting the abnormal value based on the detection signals. The indicator value 231 is the same as the case where the abnormal value 301 is detected by the second method of the above embodiment, and is the analysis result (the variance v, the standard deviation s, or the coefficient of variation cv of the signal intensity ratio PI) of the time variation analysis on the signal intensity ratio PI. Then, the control device 20A detects the body motion artifact based on the calculated indicator value 231.

The control device 20A displays the oxygen saturation at the current time or any time in the past of the oxygen saturation data group 300 on the oxygen saturation display 42 and displays the analysis result of the time variation analysis on the same time on the body motion indicator display 43 based on an operation of the user. In the example in FIG. 14, the coefficient of variation cv of the signal intensity ratio PI is, for example, a maximum value or an average value of the coefficient of variation PICV_{red} of the red light signal intensity ratio PI_{red}, the coefficient of variation PICVᵢᵣ of the infrared light signal intensity ratio PIᵢᵣ, and the coefficient of variation PICV_{grn} of the green light signal intensity ratio PI_{grn}.

When displaying the oxygen saturation display 42 and the body motion indicator display 43, the control device 20A determines whether the body motion artifact has been detected based on the analysis result at the time of display, and displays, when the body motion has been detected, the analysis result on the body motion indicator display 43 in a display format different from a case where no body motion has been detected.

For example, in a state where the coefficient of variation cv of the signal intensity ratio PI falls below a predetermined threshold, the control device 20A causes the alert display 44 (body motion display image) to display a character or a symbol regarding a possibility of the abnormal value 301 due to the body motion, and sets the display of the body motion indicator display 43 to a display format different from a state where the coefficient of variation cv exceeds the predetermined threshold. At this time, the oxygen saturation display 42 may also be displayed in a display format different from the state where the coefficient of variation cv exceeds the predetermined threshold. Differentiation of the display format in the state where the coefficient of variation cv of the signal intensity ratio PI falls below the threshold may be only one of the above three. As the difference in the display format, for example, a display format different in saturation, brightness, transparency, line type, or a combination thereof can be used. Although an example is shown in which the coefficient of variation cv of the signal intensity ratio PI is displayed on the body motion indicator display 43 as described above, the standard deviation s or the variance v may be used as the time variation analysis result.

### Functions and Effects of Embodiment

The pulse oximeter 3 of the embodiment includes the sensor unit 10A including the red light sensor 12 and the infrared light sensor 13, the control device 20A that processes the detection signal of each color, and the display unit 40 that displays the information obtained by the control device 20A. The control device 20A measures the oxygen saturation of the subject 9 based on the detection signals from the red light sensor 12 and the infrared light sensor 13 to generate the oxygen saturation data group 300, calculates the analysis result of the time variation analysis on at least one of the red light signal intensity ratio PI_{red} and the infrared light signal intensity ratio PIᵢᵣ as the indicator value 231, displays the oxygen saturation and the analysis result of the time variation analysis on the display unit 40, and displays the oxygen saturation or the analysis result in different display formats when the analysis result is equal to or more than the predetermined threshold or when the analysis result is less than the threshold.

In the pulse oximeter 3, the red light sensor 12, the infrared light sensor 13, and the control device 20A can measure the oxygen saturation of the subject 9 and display a measurement result on the display unit 40. The control device 20A can cause the user to recognize reliability of the measurement result by displaying the oxygen saturation or the analysis result in different display formats depending on whether the analysis result of the time variation analysis exceeds the threshold or falls below the threshold.

When the analysis result is less than the threshold, the pulse oximeter 3 of the embodiment changes a display color or a character format of the oxygen saturation displayed on the oxygen saturation display 42 or the analysis result displayed on the body motion indicator display 43.

Accordingly, the user can more easily grasp the body motion of the subject 9 based on the change in the display color or the character format of the oxygen saturation or the analysis result displayed on the display unit 40.

When the analysis result is less than the threshold, the pulse oximeter 3 of the embodiment displays the alert display 44 (body motion display image) indicating that there is a body motion on the display unit 40.

Accordingly, the user can grasp the body motion of the subject 9 by checking the alert display 44.

### Modifications

The disclosure is not limited to the embodiments described above, and modifications and the like within a range where the object of the disclosure can be obtained are contained in the disclosure.

The disclosure is not limited to the reflective pulse oximeter described in the embodiments, and can also be applied to a transmissive pulse oximeter. That is, the sensor unit 10 may include a red light sensor that irradiates the subject with red light from a red light source and detects red light transmitted through the subject, an infrared light sensor that irradiates the subject with infrared light from an infrared light source and detects red light transmitted through the subject, and a green light sensor that irradiates the subject with green light from a green light source and detects green light transmitted through the subject.

In the above embodiment, the pulse oximeter that measures the blood oxygen saturation has been described, and it is needless to say that the disclosure can be used not only in a device called a pulse oximeter but also in a blood oxygen wellness apparatus that displays a measurement result as a “blood oxygen level”. It should be understood that the pulse oximeter includes the blood oxygen wellness apparatus in this specification.

In the above embodiment, an apparatus for measuring the oxygen saturation of blood has been shown as an example, and the disclosure can be applied to other optical living body measurement for detecting the body motion artifact based on analysis on the signal intensity ratio PI. For example, the disclosure may be applied to an apparatus that measures an arterial blood glucose concentration.

In the embodiments described above, the configuration in which the green light sensor 11, the red light sensor 12, and the infrared light sensor 13 are provided as the sensor unit 10 is shown as an example, and as described above, the detection signals from the red light sensor 12 and the infrared light sensor 13 are used to measure the oxygen saturation. Therefore, the green light sensor 11 may not be provided.

However, by providing the green light sensor 11, it is possible to more accurately detect the body motion artifact, and it is preferable to adopt a configuration including the green light sensor 11.

### Summary of Disclosure

A pulse oximeter according to a first aspect of the disclosure includes: a red light sensor configured to irradiate a subject with red light and detect the red light transmitted through or reflected from the subject; an infrared light sensor configured to irradiate the subject with infrared light and detect the infrared light transmitted through or reflected from the subject; and a control device configured to process detection signals from the red light sensor and the infrared light sensor, in which the control device includes an oxygen saturation measurement unit for generating, based on the detection signals from the red light sensor and the infrared light sensor, an oxygen saturation data group indicating an oxygen saturation of the subject, and an abnormal value detection unit for detecting an abnormal value of the oxygen saturation data based on a body motion of the subject based on at least one of a red light signal intensity ratio indicating a ratio of an amplitude of a vibration component in the detection signal output by the red light sensor to a signal intensity of the detection signal and an infrared light signal intensity ratio indicating a ratio of an amplitude of a vibration component in the detection signal output by the infrared light sensor to a signal intensity of the detection signal.

In such a pulse oximeter, the abnormal value of the oxygen saturation data based on the body motion of the subject can be detected using the signal intensity ratio of the detection signals from the red light sensor and the infrared light sensor, and oxygen saturation measurement processing can be performed with high accuracy.

The abnormal value based on the body motion can be detected without using another sensor such as an acceleration sensor. That is, the abnormal value based on the body motion can be detected using the detection signal from the red light sensor or the infrared light sensor for measuring the oxygen saturation, and thus the configuration of the pulse oximeter can be simplified as compared to a case where another configuration for detecting the body motion such as an acceleration sensor is provided.

In the pulse oximeter of this aspect, it is preferable that, when a sudden peak point whose difference from another peak value within a predetermined period is equal to or more than a predetermined value is detected in a plurality of peak values in at least one of the red light signal intensity ratio and the infrared light signal intensity ratio, the abnormal value detection unit detects, as the abnormal value, the oxygen saturation at a timing when the sudden peak point is obtained.

When the body motion of the subject occurs, a disturbance enters the detection signal, and as a result, the sudden peak point appears in the indicator value. Therefore, by detecting the sudden peak point appearing in the indicator value, that is, the peak point whose difference from another peak value within the predetermined period is equal to or more than the predetermined value among the plurality of peak values in the indicator value, the oxygen saturation at a timing when the peak point is detected can be easily detected as the abnormal value based on the body motion.

In the pulse oximeter of this aspect, it is preferable that the abnormal value detection unit detects the abnormal value of the oxygen saturation by time variation analysis on at least one of the red light signal intensity ratio and the infrared light signal intensity ratio within a predetermined time range centered on a predetermined timing.

When the body motion of the subject occurs, a disturbance enters the detection signal, and thus the sudden peak value appears in the indicator value as described above. By performing the time variation analysis on the indicator value, the occurrence of such a sudden peak value can be easily detected, and accordingly, the abnormal value based on the body motion can be easily detected.

In the pulse oximeter of this aspect, it is preferable that the abnormal value detection unit calculates, as the time variation analysis, a standard deviation or a variance of at least one of the red light signal intensity ratio and the infrared light signal intensity ratio at n points based on the detection signals at the n points output within the predetermined time range, and detects the abnormal value based on the standard deviation or the variance.

When the variance or the standard deviation increases in the signal intensity ratio variation in the predetermined period, it means that there is a sudden peak value in the predetermined period. Therefore, presence or absence of the sudden peak value can be accurately detected by the variance or the standard deviation.

In the pulse oximeter of this aspect, it is more preferable that, based on the detection signals at n points output within the predetermined time range, the abnormal value detection unit calculates, as the time variation analysis, at least one of a coefficient of variation for the red light signal intensity ratio obtained by normalizing a standard deviation of the red light signal intensity ratio at the n points by an average value of the red light signal intensity ratio at the n points and a coefficient of variation for the infrared light signal intensity ratio obtained by normalizing a standard deviation of the infrared light signal intensity ratio at the n points by an average value of the infrared light signal intensity ratio at the n points, and detects, as the abnormal value, the oxygen saturation data at the predetermined timing when the coefficient of variation is equal to or more than a predetermined threshold.

The coefficient of variation cv of the signal intensity ratio PI can be used as a consistent detection indicator regardless of a difference between devices of the individual pulse oximeters 1 or a difference between measurement sites. Therefore, in abnormal value detection based on the coefficient of variation cv (PICV_{red}, PICVᵢᵣ, and PICV_{grn}), the abnormal value can be detected with high accuracy regardless of the differences between pulse oximeters and measurement sites.

A pulse oximeter of a second aspect of the disclosure includes: a red light sensor configured to irradiate a subject with red light and detect the red light transmitted through or reflected from the subject; an infrared light sensor configured to irradiate the subject with infrared light and detect the infrared light transmitted through or reflected from the subject; a control device configured to process detection signals from the red light sensor and the infrared light sensor; and a display unit configured to display information obtained by the control device, in which the control device generates, based on the detection signals from the red light sensor and the infrared light sensor, an oxygen saturation data group indicating an oxygen saturation of the subject, calculates an indicator value based on at least one of a red light signal intensity ratio indicating a ratio of an amplitude of a vibration component in the detection signal output by the red light sensor to a signal intensity of the detection signal and an infrared light signal intensity ratio indicating a ratio of an amplitude of a vibration component in the detection signal output by the infrared light sensor to a signal intensity of the detection signal, and displays, on the display unit, a graph in which the oxygen saturation data group and the indicator value are displayed on the same time axis.

In such a pulse oximeter, the oxygen saturation data group of the blood oxygen saturation of the subject is obtained by the red light sensor, the infrared light sensor, and the control device. In addition, the control device displays the indicator value for detecting the abnormal value based on the body motion on the display unit together with the oxygen saturation data group. Accordingly, the user can easily grasp a time when the body motion of the subject occurs based on the indicator value.

In the pulse oximeter of this aspect, it is preferable that the control device estimates a period in which a body motion of the subject occurs based on the indicator value, and superimposedly displays the period in which the body motion occurs on the graph.

Accordingly, even when the user has no knowledge about the indicator value, the user can easily check a period during which the body motion of the subject has occurred by displaying, on the display unit, an occurrence period of the body motion estimated based on the abnormal value.

In the pulse oximeter of this aspect, it is preferable that the control device uses, as the indicator value, at least one of the red light signal intensity ratio and the infrared light signal intensity ratio, and displays, superimposedly on the graph, a peak point image indicating a sudden peak point whose difference from another peak value within a predetermined period is equal to or more than a predetermined value in a plurality of peak values in the indicator value.

Accordingly, the user can easily grasp the time when the body motion of the subject occurs and the indicator value at that time.

In the pulse oximeter of this aspect, it is preferable that the control device performs time variation analysis on at least one of the red light signal intensity ratio and the infrared light signal intensity ratio within a predetermined time range centered on a predetermined timing, and displays, superimposedly on the graph, an analysis result of the time variation analysis as the indicator value.

As described above, in addition to the sudden peak point in the indicator value, it is also possible to grasp the body motion of the subject using the time variation analysis on the indicator value. Therefore, by displaying the analysis result of the time variation analysis on the indicator value on the display unit in this way, the user can grasp the time when the body motion of the subject occurs, similarly to the above-described aspect.

A pulse oximeter according to a third aspect of the disclosure includes: a red light sensor configured to irradiate a subject with red light and detect the red light transmitted through or reflected from the subject; an infrared light sensor configured to irradiate the subject with infrared light and detect the infrared light transmitted through or reflected from the subject; a control device configured to process detection signals from the red light sensor and the infrared light sensor; and a display unit configured to display information obtained by the control device, in which the control device calculates an oxygen saturation of the subject based on the detection signals from the red light sensor and the infrared light sensor, calculates, as an indicator value, an analysis result of time variation analysis on at least one of a red light signal intensity ratio indicating a ratio of an amplitude of a vibration component in the detection signal output by the red light sensor to a signal intensity of the detection signal and an infrared light signal intensity ratio indicating a ratio of an amplitude of a vibration component in the detection signal output by the infrared light sensor to a signal intensity of the detection signal, and displays the oxygen saturation and the analysis result on the display unit, and displays the oxygen saturation or the analysis result in different display formats when the analysis result is equal to or more than a predetermined threshold or when the analysis result is less than the threshold.

In such a pulse oximeter, the oxygen saturation of the subject is obtained by the red light sensor, the infrared light sensor, and the control device. The control device displays, on the display unit, the analysis result of the time variation analysis on the indicator value for detecting the body motion of the subject together with the oxygen saturation, and thus the user can easily determine the body motion of the subject.

In the pulse oximeter of this aspect, it is preferable that a display color or a character format of the oxygen saturation or the analysis result is changed when the analysis result is less than the threshold.

Accordingly, the user can more easily grasp the body motion of the subject based on the change in the display color or the character format of the oxygen saturation or the analysis result displayed on the display unit.

In the pulse oximeter of this aspect, it is preferable that a body motion display image indicating that there is a body motion is displayed when the analysis result is less than the threshold.

Accordingly, the user can more easily grasp the body motion of the subject by checking the body motion display image.

What is claimed is:

1. A pulse oximeter comprising:
a red light sensor configured to irradiate a subject with red light and detect the red light transmitted through or reflected from the subject;
an infrared light sensor configured to irradiate the subject with infrared light and detect the infrared light transmitted through or reflected from the subject; and
a control device configured to process detection signals from the red light sensor and the infrared light sensor, wherein
the control device includes
an oxygen saturation measurement unit for generating, based on the detection signals from the red light sensor and the infrared light sensor, an oxygen saturation data group indicating an oxygen saturation of the subject, and
an abnormal value detection unit for detecting an abnormal value of the oxygen saturation data based on a body motion of the subject based on at least one of a red light signal intensity ratio indicating a ratio of an amplitude of a vibration component in the detection signal output by the red light sensor to a signal intensity of the detection signal and an infrared light signal intensity ratio indicating a ratio of an amplitude of a vibration component in the detection signal output by the infrared light sensor to a signal intensity of the detection signal.

2. The pulse oximeter according to claim 1, wherein
when a sudden peak point whose difference from another peak value within a predetermined period is equal to or more than a predetermined value is detected in a plurality of peak values in at least one of the red light signal intensity ratio and the infrared light signal intensity ratio, the abnormal value detection unit detects, as the abnormal value, the oxygen saturation at a timing when the sudden peak point is obtained.

3. The pulse oximeter according to claim 1, wherein
the abnormal value detection unit detects the abnormal value of the oxygen saturation by time variation analysis on at least one of the red light signal intensity ratio and the infrared light signal intensity ratio within a predetermined time range centered on a predetermined timing.

4. The pulse oximeter according to claim 3, wherein
the abnormal value detection unit calculates, as the time variation analysis, a standard deviation or a variance of at least one of the red light signal intensity ratio and the infrared light signal intensity ratio at n points based on the detection signals at the n points output within the predetermined time range, and detects the abnormal value based on the standard deviation or the variance.

5. The pulse oximeter according to claim 3, wherein
based on the detection signals at n points output within the predetermined time range, the abnormal value detection unit calculates, as the time variation analysis, at least one of a coefficient of variation for the red light signal intensity ratio obtained by normalizing a standard deviation of the red light signal intensity ratio at the n points by an average value of the red light signal intensity ratio at the n points and a coefficient of variation for the infrared light signal intensity ratio obtained by normalizing a standard deviation of the infrared light signal intensity ratio at the n points by an average value of the infrared light signal intensity ratio at the n points, and detects, as the abnormal value, the oxygen saturation data at the predetermined timing when the coefficient of variation is equal to or more than a predetermined threshold.

6. A pulse oximeter comprising:
a red light sensor configured to irradiate a subject with red light and detect the red light transmitted through or reflected from the subject;
an infrared light sensor configured to irradiate the subject with infrared light and detect the infrared light transmitted through or reflected from the subject;
a control device configured to process detection signals from the red light sensor and the infrared light sensor; and
a display unit configured to display information obtained by the control device, wherein
the control device
generates, based on the detection signals from the red light sensor and the infrared light sensor, an oxygen saturation data group indicating an oxygen saturation of the subject,
calculates an indicator value based on at least one of a red light signal intensity ratio indicating a ratio of an amplitude of a vibration component in the detection signal output by the red light sensor to a signal intensity of the detection signal and an infrared light signal intensity ratio indicating a ratio of an amplitude of a vibration component in the detection signal output by the infrared light sensor to a signal intensity of the detection signal, and
displays, on the display unit, a graph in which the oxygen saturation data group and the indicator value are displayed on the same time axis.

7. The pulse oximeter according to claim 6, wherein
the control device estimates a period in which a body motion of the subject occurs based on the indicator value, and superimposedly displays the period in which the body motion occurs on the graph.

8. The pulse oximeter according to claim 6, wherein
the control device uses, as the indicator value, at least one of the red light signal intensity ratio and the infrared light signal intensity ratio, and displays, superimposedly on the graph, a peak point image indicating a sudden peak point whose difference from another peak value within a predetermined period is equal to or more than a predetermined value in a plurality of peak values in the indicator value.

9. The pulse oximeter according to claim 6, wherein
the control device performs time variation analysis on at least one of the red light signal intensity ratio and the infrared light signal intensity ratio within a predetermined time range centered on a predetermined timing, and displays, superimposedly on the graph, an analysis result of the time variation analysis as the indicator value.

10. A pulse oximeter comprising:
a red light sensor configured to irradiate a subject with red light and detect the red light transmitted through or reflected from the subject;
an infrared light sensor configured to irradiate the subject with infrared light and detect the infrared light transmitted through or reflected from the subject;
a control device configured to process detection signals from the red light sensor and the infrared light sensor; and
a display unit configured to display information obtained by the control device, wherein
the control device
calculates an oxygen saturation of the subject based on the detection signals from the red light sensor and the infrared light sensor,
calculates, as an indicator value, an analysis result of time variation analysis on at least one of a red light signal intensity ratio indicating a ratio of an amplitude of a vibration component in the detection signal output by the red light sensor to a signal intensity of the detection signal and an infrared light signal intensity ratio indicating a ratio of an amplitude of a vibration component in the detection signal output by the infrared light sensor to a signal intensity of the detection signal, and
displays the oxygen saturation and the analysis result on the display unit, and displays the oxygen saturation or the analysis result in different display formats when the analysis result is equal to or more than a predetermined threshold or when the analysis result is less than the threshold.

11. The pulse oximeter according to claim 10, wherein
a display color or a character format of the oxygen saturation or the analysis result is changed when the analysis result is less than the threshold.

12. The pulse oximeter according to claim 10, wherein
a body motion display image indicating that there is a body motion is displayed when the analysis result is less than the threshold.
